(19) United States
(12) Patent Application Publication
(10) Pub. No.: US 2012/0065987 A1
(43) Pub. Date: Mar. 15, 2012

(54) Computer-Based Patient Management for Healthcare

(75) Inventors: Faisal Farooq, Norristown, PA (US); Romer E. Rosales, Downingtown, PA (US); Shipeng Yu, Exton, PA (US); Balaji Krishnapuram, King of Prussia, PA (US); Bharat R. Rao, Berwyn, PA (US)

(73) Assignee: SIEMENS MEDICAL SOLUTIONS USA, INC., Malvern, PA (US)

(21) Appl. No.: 13/228,497

(22) Filed: Sep. 9, 2011

Related U.S. Application Data
(63) Continuation-in-part of application No. 13/153,551, filed on Jun. 6, 2011.
(60) Provisional application No. 61/381,084, filed on Sep. 9, 2010.

(51) Int. Cl. G06Q 50/22

(52) U.S. Cl. 705/2

(57) ABSTRACT

Computer-based patient management is provided for healthcare. Patient data is used to determine a severity, assign a patient to a corresponding diagnosis-related group, and provide a timeline for care at a medical facility. Reminders or alerts are sent to maintain the timeline for more cost effective care. Reminders, suggestions, transitions between care givers, scheduling and other risk management actions are performed. As more data becomes available as part of the care, the care and timeline may be adjusted automatically for more efficient utilization of resources. Accountable care optimization is provided as part of case management. Automated care before any injury or illness and automated care after discharge is provided to optimize the health and costs for a patient. The patient is assigned to the cohort based on the patient data.

### RELATED APPLICATIONS

The present patent document is a continuation-in-part of U.S. patent application Ser. No. 13/153,551, filed Jun. 6, 2011 and claims the benefit of the filing date under 35 U.S.C. §119(e) of Provisional U.S. patent application Ser. No. 61/381,084, filed Sep. 9, 2010, which are hereby incorporated by reference.

### BACKGROUND

The present embodiments relate to a computerized system for case management or accountable care optimization.

According to the American Case Management Association (ACMA), the case management process encompasses communication and facilitates care along a continuum through effective resource coordination. The goals of case management include the achievement of optimal health, access to care and appropriate utilization of resources, balanced with the patient's right to self determination. The goals of case management are to facilitate timely discharges, prompt and efficient use of resources, achievement of expected outcomes, and performance improvement activities which lead to optimal patient outcomes.

In a typical setting, a healthcare facility hires personnel (e.g. case managers or case management nurses) that typically fulfill roles of utilization review manager, quality manager, or discharge planner. These case managers review charts for the use of interdependent hospital systems, timeliness of service as well as safe and appropriate utilization of services. The case managers work with a physician for monitoring the quality of services provided to the patient. For example, high-risk patients with high-risk diagnosis (e.g., stroke, myocardial infarction, or complicated pneumonia) are evaluated. If after review of the patient's stay and utilization of services, a patient no longer needs to stay in an acute care setting, the case manager may request of the attending physician that the patient have outpatient or utilize other services. The evaluation may not only impact quality of care and patient outcome, but also may have financial and legal implications for healthcare facilities. Financial implications exist for low risk patients as well. The sooner a low risk patient is discharged from the hospital, the higher the rates of reimbursement can be.

The case manager evaluates some, but not all patients. To perform case management, the case manager manually reviews charts and clinical records for patients and in turn formulates care plans, assigns patients to diagnosis-related groups (DRG), and creates a discharge timeline. Due to the laborious nature of the task, typically case management is done for a random sample of patients. In some institutions, computerized tools present data for a patient in a unified manner. The computerized tools merely provide for a simpler presentation of data and rely on the case manager for action. These tools may fail to fully improve the actual patient level outcome and may fail to substantially increase the number of patients evaluated.

### SUMMARY

In various embodiments, systems, methods and computer readable media are provided for computer-based patient management for healthcare. Case management is provided by a processor with or without further management by a person. Patient data is used to determine a severity, assign a patient to a corresponding diagnosis-related group, and/or provide a timeline for care. Reminders or alerts are sent to maintain the timeline for more cost effective care. As more data becomes available as part of the care, the care and timeline may be adjusted automatically for more efficient utilization of resources.

The case management is performed for a patient stay at a medical facility. The case management may additionally be performed outside the medical facility. Accountable care optimization is provided as part of case management. Automated care management before any injury or illness and automated care management after discharge are provided to optimize the health and costs for a patient. Reminders, suggestions, transitions between care givers, scheduling and other risk management actions are performed based on a cohort to which a patient is assigned. The patient is assigned to the cohort based on the patient data.

In a first aspect, a method is provided for computer-based patient management for healthcare. A processor gathers first clinical data for a patient of a healthcare facility. The processor establishes a workflow for care of the patient as a function of the first clinical data and a cost factor. The workflow is for multiple actions by different entities of the healthcare facility and includes a timeline for the actions. The processor obtains second clinical data after the establishing and as part of the workflow for the care of the patient. The processor updates the workflow for the care of the patient as a function of the first and second clinical data and the cost factor. The updating occurs while the patient is at the healthcare facility. The processor generates at least one alert for at least one of the multiple actions. The alert is generated as a function of the timeline.

In a second aspect, a system is provided for computer-based patient management for healthcare. At least one memory is operable to store data for a plurality of patients. A first processor is configured to classify each of the patients into diagnosis-related groups based on respective data for each of the patients, select a timeline to discharge as a function of the diagnosis-related group for each of the patients, alter the diagnosis-related group for at least one of the patients, the altering being based on a utilization and new data not used in the classifying, change the timeline for the one of the patients, the changing being a function of the altering, and monitor tasks across multiple medical professionals as a function of the timeline.

In a third aspect, a non-transitory computer readable storage medium has stored therein data representing instructions executable by a programmed processor for computer-based patient management for healthcare. The storage medium includes instructions for acquiring data for a patient, establishing, as a function of the data, first care for the patient prior to an admission to a healthcare facility, managing, as a function of the data, second care for the patient upon the admission to the healthcare facility, and establishing, as a function of the data, third care for the patient after discharge from the healthcare facility.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

FIG. 1 is a flow chart diagram of one embodiment of a method for computer-based patient management for healthcare;

FIG. 2 is a flow chart diagram of one embodiment of a method for computer-based accountable care optimization for healthcare;

FIG. 3 shows an exemplary data mining framework for mining clinical information;

FIG. 4 shows an exemplary computerized patient record (CPR);

FIG. 5 shows a block diagram of a system for patient management for healthcare according to one embodiment; and

FIG. 6 is another exemplary data mining framework for mining in computer-based patient management for healthcare.

### DESCRIPTION OF PREFERRED EMBODIMENTS

Case management is automated at healthcare facilities and/or for a patient before, after and/or during a stay at a healthcare facility. The severity of an illness or injury, diagnosis-related group, and/or a cohort to which a patient belongs is predicted from data for the patient. For case management, patient data is obtained or mined from electronic medical records (EMRs), such as patient information databases, radiology information systems (RIS), pharmacological records, or other form of medical data storage or representation. In an EMR or RIS, various data elements are normally associated to a patient or patient visit, such as diagnosis codes, lab results, pharmacy, insurance, doctor notes, images, and genotypic information.

The system combines information from multiple sources and produces the best DRG considering the symptoms, severity, all morbidities and co-morbidities and by presenting the evidence for such an output. This is valuable for multiple reasons i) it may provide a better care plan for the patient since all the relevant information is mined and presented some of which may have been missed by any manual review, ii) the financial outcomes are better for care providers because the manual process could classify the patient into a lesser severity group than it actually belongs to which results in lesser payments iii) the denial of claims may be minimal as for all DRGs, the evidence for including that group is clearly presented as a part of the mining process.

Using the mined data, a computer system predicts the severity of an illness or injury and/or class to which a patient belongs for treatment. Based on the prediction and cost considerations, a workflow and/or timeline to care for the patient are created. Clinical records for a patient are combined with the clinical knowledge and case management guidelines to automatically perform the tasks for case management. Schedules, reminders, alerts, or other tasks are created and monitored to manage the care of the patient while optimizing utilization.

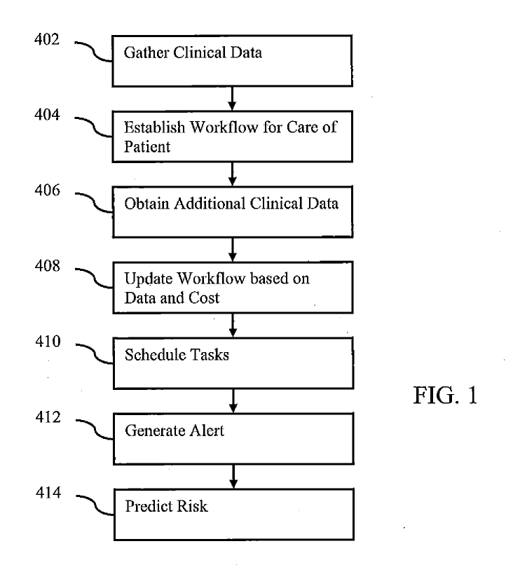

FIG. 1 shows a method for computer-based patient management for healthcare at a healthcare facility. The method is implemented by or on a computer, server, workstation, system, or other device. The method is provided in the order shown, but other orders may be provided. Additional, different or fewer acts may be provided. For example, act 414 may not be provided.

Continuous (real time) or periodic classification of the diagnosis-related group and/or severity is performed. Throughout a stay at a hospital or other healthcare facility, the care is tuned based on the most recent data and associated classification. The care of the patient is managed based on the current status of the patient derived from patient specific data and based on cost or other utilization considerations. As the time passes and as more data (e.g., new labs results, new medications, new procedures, existing history etc.) is gathered, the care plan for the patient may be updated and presented to a case or patient manager.

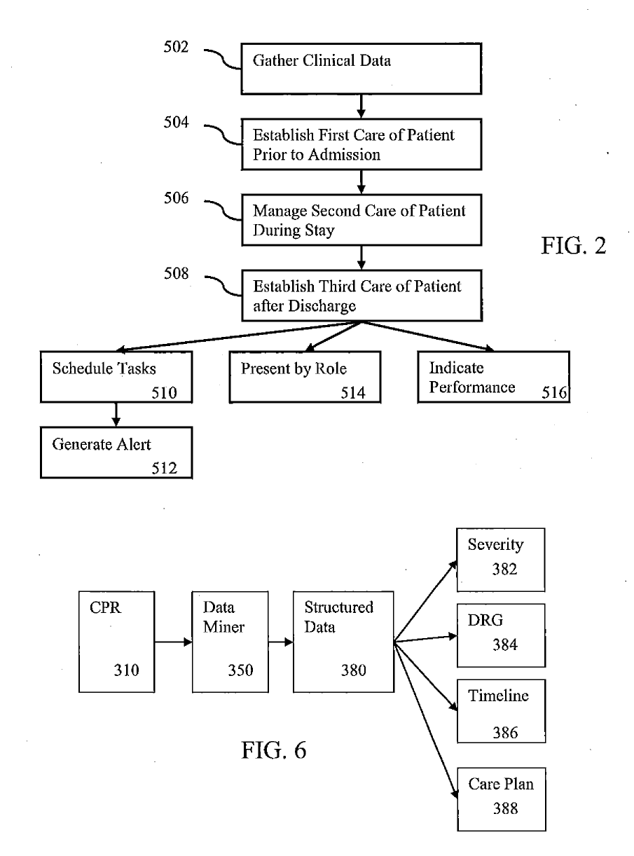

FIG. 1 is directed to case management at a healthcare facility. Healthcare facilities include hospitals, emergency care centers, or other locations or organizations for treating illness or injury. The patient may stay one or more days at a healthcare facility for diagnosis and/or treatment. In some cases, the stay may be only hours. FIG. 2 is directed to accountable care optimization, which may or may not include care at a healthcare facility. The care of the patient before, during, and after any stay at a healthcare facility is managed. Given the rise in accountable care where the care provider shares the financial risk, managing care before a stay, during a stay, and after the stay based on patient well being and associated cost considerations allows alteration of the care of the patient in such a way that the cost of care is kept low. For example, the care of the patient at a healthcare facility may be different (e.g., perform an extra task, such as education) in order to reduce costs for later care of the patient outside the healthcare facility or after discharge. It may also prevent an unplanned readmission of the patient which results in increased cost and often penalties by payers such as CMS. In both FIGS. 1 and 2, a computer performs the case management and/or presents options to a case manager for the management of care.

Referring again to FIG. 1, the case management operation is triggered in response to an event. For example, an indication of admission of a patient to a healthcare facility or new data for the patient being available is received. The receipt is by a computer or processor. For example, a nurse or administrator enters data for the medical record of a patient. The data entry indicates admission to the healthcare facility, to a practice group within the healthcare facility or to a different practice. Similarly, indication of transfer or discharge to another practice group, facility, or practice may be received. As another example, a new data entry is provided in the electronic medical record of the patient. In another example, an assistant enters data showing a key trigger event (e.g., completion of surgery, assignment of the patient to another care group, completion in a task of the workflow for care of the patient, or a change in patient status). In alternative embodiments, the indication is not received and periodic or continuous operation is provided.

In response to the trigger, an automated workflow is started. The indication or other trigger causes a processor to run a case management process. The case management workflow determines a cohort or diagnostic-related group for the patient and then establishes a workflow of care for the patient.

In act 402, clinical data about a patient is gathered. The case management workflow includes establishing the care for the patient. To establish the workflow of care, the case management workflow first gathers data for the patient.

A processor gathers clinical data for a patient of a healthcare facility. The data is gathered by searching or by loading from the medical record. In other embodiments, the information to be used for establishing the workflow of care for the patient is not available as specific values in the medical record or inconsistent data is provided. Rather than merely searching or loading data, the electronic medical record of the patient may be mined. Mining combines local and/or global evidence from medical records with the medical knowledge and guidelines to make inferences over time. Local evidence may include information available at the healthcare facilities, and global evidence may include information available from other sources, such as other healthcare facilities, insurance companies, primary care physicians, or treating physicians.

The classifier for case management has an input feature vector or group of variables used for establishing a workflow for care. The values for the variables for a particular patient are obtained by mining the electronic medical record for the patient. The electronic medical record for the patient is a single database or a collection of databases. The record may include data at or from different medical entities, such as data from a database for a hospital and data from a database for a primary care physician whether affiliated or not with the hospital. Data for a patient may be mined from different hospitals. Different databases at a same medical entity may be mined, such as mining a main patient data system, a separate radiology system (e.g., picture archiving and communication system), a separate pharmacy system, a separate physician notes system, and/or a separate billing system. Different data sources for the same and/or different medical entities are mined.

The different data sources have a same or different format. The mining is configured for the formats. For example, one, more, or all of the data sources are of structured data. The data is stored as fields with defined lengths, text limitations, or other characteristics. Each field is for a particular variable. The mining searches for and obtains the values from the desired fields. As another example, one, more, or all of the data sources are of unstructured data. Images, documents (e.g., free text), or other collections of information without defined fields for variables is unstructured. Physician notes may be grammatically correct, but the punctuation does not define values for specific variables. The mining may identify a value for one or more variables by searching for specific criteria in the unstructured data.

Any now known or later developed mining may be used. For example, the mining is of structured information. A specific data source or field is searched for a value for a specific variable. As another example, the values for variables are inferred. The values for different variables are inferred by probabilistic combination of probabilities associated with different possible values from different sources. Each possible value identified in one or more sources are assigned a probability based on knowledge (e.g., statistically determined probabilities or professionally assigned probabilities). The possible value to use as the actual value is determined by probabilistic combination. The possible value with the highest probability is selected. The selected values are inferred values for the variables of the feature vector of the classifier for case management.

U.S. Pat. No. 7,617,078, the disclosure of which is incorporated herein by reference, shows a patient data mining method for combining electronic medical records for drawing conclusions. This system includes extraction, combination and inference components. The data to be extracted is present in the hospital electronic medical records in the form of clinical notes, procedural information, history and physical documents, demographic information, medication records or other information. The system combines local and global (possibly conflicting) evidences from medical records with medical knowledge and guidelines to make inferences over time. Existing knowledge, guidelines, best practices, or institution specific approaches are used to combine the extracted data for case management.

U.S. Published Application No. 2003/0120458, the disclosure of which is incorporated herein by reference, discloses mining unstructured and structured information to extract structured clinical data. Missing, inconsistent or possibly incorrect information is dealt with through assignment of probability or inference. These mining techniques are used for quality adherence (U.S. Published Application No. 2003/0125985), compliance (U.S. Published Application No. 2003/0125984), clinical trial qualification (U.S. Published Application No. 2003/0130871), and billing (U.S. Published Application No. 2004/0172297). The disclosures of the published applications referenced in the above paragraph are incorporated herein by reference. Other patent data mining or mining approaches may be used, such as mining from only structured information, mining without assignment of probability, or mining without inferring for inconsistent, missing or incorrect information. In alternative embodiments, values are input by a user for applying the predictor without mining.

In act 404, the processor establishes a workflow for care of the patient. The workflow of care is established as a function of the gathered clinical data and one or more cost factors. Alternatively, the workflow of care is established as a function of the clinical data without specific cost factors. The workflow of care itself is based on clinical guidelines, hospital treatment standards, or other sources.

The clinical data is used to predict a severity and assign the patient to a diagnosis-related group as a function of the severity. For example, based on the past and current medical records of a patient, the patient is classified into a diagnosis-related group. Diagnosis-related groups are groups of patients to receive the same care, such as all acute myocardial infarction patients being in one group. For example, the patient is assigned to one of five types of myocardial infarction. Greater or lesser grouping may be provided, such as providing a single myocardial infarction group.

The severity may indicate the appropriate diagnostic-related group. By quantifying severity (e.g., low, medium and high), the specific diagnostic-related group may be determined. The severity may reflect the presence of complications or co-morbidities, resulting in a different diagnostic-related group (e.g., acute myocardial infarction in patients with diabetes being a different group than acute myocardial infarction). In alternative embodiments, the diagnostic-related group is established independently of severity.

To classify the patient into a diagnostic-related group and/or predict severity, the gathered clinical data is applied to a classifier or model. In one embodiment, different classifiers are provided for the respective different diagnosis-related groups and/or severities. In other embodiments, a single classifier distinguishes between different diagnosis-related groups and/or severities. For example, the classifier clusters based on the clinical data.

A probability of the patient being in each given class is output. The class associated with the highest probability is selected for the patient. In other embodiments, the classifier determines the class without a probability. Manually programmed criteria may be applied to distinguish among classes. In one embodiment, a machine-learned classifier uses the patient data to establish the workflow for care of the patient or at least provides severity and/or diagnosis-related grouping to be used for establishing the workflow for care.

A feature vector used for classifying is populated. By mining, the values for variables are obtained. The feature vector is a list or group of variables used to classify. The mining outputs values for the feature vector. The output is in a structured format. The data from one or more data sources, such as an unstructured data source, is mined to determine values for specific variables. The values are in a structured format—values for defined fields are obtained.

The mining may provide all of the values, such as resolving any discrepancies based on probability. Any missing values may be replaced with an average or predetermined value. The user may be requested to enter a missing value or resolve a choice between possible values for a variable. Alternatively, missing values are not replaced where the classifier may operate with one or more of the values missing.

The feature vector is populated by assigning values to variables in a separate data storage device or location. A table formatted for use by the classifier is stored. Alternatively, the values are stored in the data sources from which they are mined and pointers indicate the location for application of the classifier.

The diagnosis-related group and/or severity class is provided by applying the classifier. In one embodiment, the classifier is a machine-trained classifier. Any machine training may be used, such as training a statistical model (e.g., Bayesian network). The machine-trained classifier is any one or more classifiers. A single class or binary classifier, collection of different classifiers, cascaded classifiers, hierarchal classifier, multi-class classifier, model-based classifier, classifier based on machine learning, or combinations thereof may be used. Multi-class classifiers include CART, K-nearest neighbors, neural network (e.g., multi-layer perceptron), mixture models, or others. A probabilistic boosting tree may be used. Error-correcting output code (ECOC) may be used. In one embodiment, the machine-trained classifier is a probabilistic boosting tree (PBT) classifier. The detector is a tree-based structure with which the posterior probabilities of class membership are calculated from given values of variables. The nodes in the tree are constructed by a nonlinear combination of simple classifiers using boosting techniques. The PBT unifies classification, recognition, and clustering into one treatment. Alternatively, a programmed, knowledge based, or other classifier without machine learning is used.

For learning-based approaches, the classifier is taught to distinguish based on features. For example, a probability model algorithm selectively combines features into a strong committee of weak learners based on values for available variables. As part of the machine learning, some variables are selected and others are not selected. Those variables with the strongest or sufficient correlation or causal relationship to a cohort, severity, or diagnosis-related group are selected and variables with little or no correlation or causal relationship are not selected. Features that are relevant to case management or care are extracted and learned in a machine algorithm based on the ground truth of the training data, resulting in a probabilistic model. Any size pool of features may be extracted, such as tens, hundreds, or thousands of variables. The pool is determined by a programmer and/or may include features systematically determined by the machine. The training determines the most determinative features for a given classification and discards lesser or non-determinative features.

The classifier is trained from a training data set using a computer. To prepare the set of training samples, actual severity, diagnosis-related group, or cohort is determined for each sample (e.g., for each patient represented in the training data set). Any number of medical records for past patients is used. By using example or training data for tens, hundreds, or thousands of examples with known status, a processor may determine the interrelationships of different variables to the outcome. The training data is manually acquired or mining is used to determine the values of variables in the training data. The training may be based on various criteria, such as readmission within a time period.

The training data is for the medical entity for which the predictor will be applied. By using data for past patients of the same medical entity, the variables or feature vector most relevant to care management for that entity are determined. Different variables may be used by a machine-trained classifier for one medical entity than for another medical entity. Some of the training data may be from patients of other entities, such as using half or more of the examples from other entities with similar workflow of care, concerns, sizes, or patient populations. The training data from the specific institution may skew or still result in a different machine-learnt classifier for the entity than using fewer examples from the specific institution. In alternative embodiments, all of the training data is from other medical entities, or the classifier is trained in common for a plurality of different medical entities.

In alternative embodiments, the predictor is programmed, such as using physician knowledge or the results of studies. Input values of variables are used by domain knowledge to classify.

The classifier is trained to class patients in general. For example, the output of the classifier is an identity of one of multiple different classes. Alternatively, separate classifiers are trained for different classes, such as training a classifier for acute myocardial infarction and another classifier for angina. Different classifiers are trained to indicate a probability of a given patient being a member of a given class. By applying the multiple classifiers, the patient is assigned to a class or combination of classes based on relative probabilities. For example, the diagnoses above a 50% probability are used to identify a diagnosis-related group representing a combination of diagnoses. Similarly, the classifier may identify co-morbidities.

The learnt predictor is a matrix. The matrix provides weights for different variables of the feature vectors. The values for the feature vector are weighted and combined based on the matrix. The classifier is applied by inputting the feature vector to the matrix. Other representations than a matrix may be used.

For application, the classifier is applied to the electronic medical record of a patient. In response to the triggering, the values of the variables used by the learned classifier are obtained. The values are input to the classifier as the feature vector. The classifier outputs a class or a probability of the patient being in a given class based on the patient's current electronic medical record.

The class is determined automatically. The user may input one or more values of variables into the electronic medical record, but the classification is performed without entry of values after the trigger and while applying the classifier. Alternatively, one or more inputs are provided, such as resolving ambiguities in values or to select an appropriate classifier (e.g., select a predictor of infection as opposed to trauma).

By applying the classifier to mined information for a patient, a probability of membership in a class is provided for that patient. The machine-learnt or other classifier outputs a statistical probability of class based on the values of the variables for the patient. Where the classification occurs in response to an event, such as triggering at the request of a medical professional or administrator, the class is provided from that time.

The classifier may indicate one or more values contributing to the probability. For example, the mention of myocardial infarction in physician notes is identified as being the strongest link or contributor to a probability of the patient being in a heart attack group. This variable and value are identified. The machine-learnt classifier may include statistics or weights indicating the importance of different variables to class membership. In combination with the values, some weighted values may more strongly determine an increased probability of membership. Any deviation from norm may be highlighted. For example, a value or weighted value of a variable a threshold amount different from the norm or mean is identified. The difference alone or in combination with the strength of contribution to the class membership is considered in selecting one or more values as more significant. The more significant value or values may be identified.

In alternative embodiments of creating and applying the classifier, the class is integrated as a variable to be mined. The inference component determines the class based on combination of probabilistic factoids or elements. The class is treated as part of the patient state to be mined. Domain knowledge determines the variables used for combining to output the class.

The classifier outputs a diagnosis-related group for the patient. Alternatively, the classifier outputs a severity. The severity, with or without other patient data, is used to determine the diagnosis-related group. For example, the patient belongs to the genus group of myocardial infarction. The severity indicates a more specific diagnosis-related group.

The case management workflow queries the results of the mining and/or classification of the patient into a diagnosis-related group or severity. The workflow uses the results or is included as part of the classifier application. Any now known or later developed software or system providing a workflow engine may be configured to initiate a workflow based on data.

To establish the workflow for care of the patient, the diagnosis-related group, severity, and/or variables associated with the class for a particular patient may be used to determine a mitigation plan. The mitigation plan includes instructions, prescriptions, education materials, schedules, clinical actions, tests, visits, examinations, or other jobs that may care for the patient. The next recommended clinical actions or reminders for the next recommended clinical actions may be output so that health care personnel are better able to follow the recommendations.

A library of workflows for care (mitigation plans) is provided. At least one workflow for care is provided for each diagnosis-related group. Separate care workflows may be provided for different diagnosis-related groups. The severity may be used to select between different workflows of care for a same diagnosis-related group. The workflow of care appropriate for a given patient is obtained and output.

The cost factor may be included in the selected workflow. For example, when the severity of the patient is predicted better due to the combination of all of the information, the workflow might suggest directly getting a CT instead of first getting an x-ray and then ordering a CT when the x-ray results are not sufficient to reach a conclusion. This would not only save an extra exam but would also cut on the length of stay. Another example would be to create the optimal path or the critical task map where it becomes evident which tests/procedures can be done without waiting on results from others and which should be done in order one after the other. This will make results available quickly and possibly save on some procedures/tests.

For a given diagnosis, the most cost effective treatment with sufficient or better outcome for the patient is used as part of the workflow for that diagnosis. Rather than just rely on best or sufficient care, the best or sufficient care with optimized cost may be used. For example, testing for diabetes in myocardial infarction patients performed early in a patient's stay may result in more optimized care later by avoiding treatments not as effective for patients with this complication. By including a test for diabetes within a first day of the patient stay as part of the workflow, a better utilization of resources may be provided. The cost is built into the workflow.

In other embodiments, the cost factor is used as a factor for selecting the workflow. Different workflows for a given diagnosis may be provided. The workflow with a lesser cost to the healthcare facility may be selected. The selection may be based only on cost factor, such as where each workflow of care is appropriate, or based on cost factor and other variables, such as relatively weighting severity, cost factor to select between care workflow with a range of successful outcome, and/or data for the patient.

In other embodiments, the classifier is trained to class based, in part, on the cost factor. For example, one or more cost factors are used as input features. As another example, the classes are defined based on cost factors, such as dividing one general class into specific classes that may be reimbursed differently.

The cost factor may be a cost of care, a reimbursement for the care, or other utilization. Workflows with a cheaper cost to the healthcare facility, such as having a nurse perform an action instead of a physician, may be selected. Follow up calls can be scheduled to make sure that the patient is taking medications or follows up with the primary care/nursing facility. The cost of a short call could avoid the cost of a possible readmission of complication due to patient non-compliance. Workflows with a higher rate of return or payment likelihood, such as a workflow avoiding non-reimbursable or experimental treatment, may be selected. Workflows with a less cost to the patient may be selected. A combination of cost factors may be used to select, in part, the workflow for care. Patient outcome, such as success rate or readmission avoidance, may be another or more greatly weighted factor for selection of the workflow of care.

In other embodiments, the case management is performed at a population or cohort level. In an accountable care setting, data is shared between the participating entities. For example, primary care providers and payers share data with the participating hospital and critical care facilities. A case manager in this setting can manage a cohort instead of a single patient. A group of patients with similar diagnoses or severities or other similar characteristics can be managed within the facility or even outside it in an accountable care setting. The patient group that is predicted to be more risky patients can be asked to follow up with participating primary care providers or nursing care facilities more often and their hospital costs are kept at a minimal. Also, alerts are generated and sent to corresponding stakeholders e.g. when a patient is discharged, the case management workflow generates an alert for a follow up appointment with the primary care provider for a physical exam within a given timeframe failing which a reminder is sent to the office or the patient. The cohort/group level management may also include cost factors such as the cohort of patients that are the most expensive for the organization and the items in the workflow that account to the majority of the costs. This can often help in optimizing the workflow by optimizing and tuning the standard care procedure to that particular organization.

In one embodiment, the case management workflow suggests particular providers for a patient or cohort, such as specialists or critical care facilities that in the past have best managed such cases. This is performed by mining the patient outcomes and cost information for all the participating providers and then correlating them with, but not limited to, the DRG and other cohort information.

In one embodiment, the system simulates multiple workflows for a patient or group of patients and provides comparative effectiveness in terms of outcomes and also comparison of cost on the different possible outcomes. The system also provides the most optimal workflow, the workflow with best patient outcome and the workflow with minimal cost. The provider can select one of the many workflows and as more data is input into the system during the care of the patient, the workflow is updated accordingly.

Case Management may include care management and optimization, risk management and optimization, financial management and optimization, and workflow management and optimization

The workflow for care includes multiple actions by different entities of the healthcare facility in a timeline for the actions. The timeline may be maximized for efficiency and/or to provide savings. The actions are for tests, treatment, consultation, discharge, transfer or other tasks performed at the healthcare facility. The actions are performed by different people, such as nurses, physicians, administrators, techs, volunteers, or others. By providing a timeline, the different people involved may be coordinated to maximize the utilization of their time and healthcare facility resources.

In one embodiment, the workflow for care of the patient is established for review and monitoring by a case manager. The workflow for care may include actions or tasks to be performed by the case manager. The task entry may be to update patient data, arrange for clinical action, update a prescription, arrange for a prescription, review test results, arrange for testing, schedule a follow-up, review the probability, review patient data, or other action to reduce the probability of readmission. For example, where a follow-up is not scheduled during discharge and is not automatically arranged, arranging for the follow-up may be placed as an action item in an administrator's, assistant's, nurse's, or other case manager's workflow. As another example, a test is included in the timeline to be ordered to provide the missing information. Review of test results is placed in a physician's workflow by the case management workflow so that appropriate action may be taken before or after other actions. Missing information from the patient data may be identified. A workflow action is automatically scheduled for a case manager to contact the physician via phone or in person to inquire about performance of the test and/or review of the results.

The case manager may review the established workflow for care and alter the tasks or timeline. The workflow may be examined to determine if other action was warranted. Future workflow action items, discharge instructions, physician education, or other actions may be performed to avoid inefficiencies or care issues in other patients.

The case management workflow schedules, monitors, or otherwise implements the workflow for care. By accessing calendars, schedules or workflows, the case management workflow causes the workflow for care to be performed in conformance with the timeline.

The timeline of the selected or established workflow may be automatically altered to account for timelines of other workflows. For example, equipment (e.g., a medical imaging system), a person (e.g., a physician), or room (e.g., an operating or treatment room), a device (e.g., a catheter) or other resource may be unavailable due to other appointment, delivery timing, work schedule, or other reason. The closest availability to optimal may be selected. Other timelines may be adjusted, such as adjusting scheduled tasks for a timeline associated with a lesser overall cost due to delay and not adjusting a timeline for a workflow of care associated with a greater overall cost due to delay.

The workflow for care may include actions, such as for the case manager, to document for reimbursement or other purposes. Tasks specific to storing or obtaining proper documentation may be included in the timeline. The patient record may be mined or searched to identify the needed documents. Where the documents are not found, the processor may add tasks to the timeline for obtaining the documentation. The tasks for documentation may be assigned to personnel responsible for the creation or to a case manager responsible for getting the personnel to provide the documentation.

The timeline indicates an optimal discharge time. This prediction may be useful to the patient or for planning tasks to occur during the stay. For cost reasons, the discharge time may be longer to allow additional tasks. Despite the increase in cost for the current care at the facility, the cost for overall care including after discharge may be reduced. This cost may be part of the workflow for care and/or is based on data specific to the patient. For example, the workflow for care is different for two patients in the same diagnosis-related group since one patient has insurance and the other does not. The patient with insurance may be more likely to visit a physician for a follow-up, so is discharged earlier. The patient without insurance stays longer according to the workflow to allow follow-up. A given workflow for care may have data driven branches for tasks (job paths) or different workflows may be used.

In act 406, the processor obtains additional clinical data. The additional clinical data is obtained after establishing the workflow for care. The additional data may be generated as part of the workflow for the care of the patient. For example, the workflow includes a physician visit or diagnosis. The physician creates notes including the diagnosis where the notes are added to the patient record, or the physician enters information into a diagnosis system. As another example, a test is performed and the results are added to the patient record. By performing one or more of the actions in the workflow for care, additional clinical data is generated. The additional data may be acquired from actions not in the workflow for care.

In other embodiments, the additional clinical data was acquired before establishing the workflow for care, but was not previously available. The later availability results in the clinical data being additional data. Using a flag, trigger, scan, or other mechanism, the additional data or addition of additional data is detected by the processor. For example, any additional clinical data entered into the patient record triggers a review of the workflow. The case management workflow is triggered to review the workflow of care.

In act 408, the processor updates the workflow for the care of the patient. The update changes or replaces the current workflow with another workflow.

The update is performed as a function of the clinical data and the cost factor. For example, the same process for establishing the workflow is performed. In addition to the original patient data, the additional patient data is used. The additional patient data may indicate different values for variables (e.g., change from smoker to non-smoker due to further evidence). The different value or values may result in a different classification. For example, the severity is changed, resulting in a different diagnosis-related group for the patient. The workflow for the patient is reassigned due to the difference in severity. In alternative embodiments, the additional data is used to change any affected values without re-obtaining all of the values. In other embodiments, the additional data itself is used to identify any change to the workflow without reapplying the classifier and/or mining.

The case management workflow identifies any tasks or jobs in the updated workflow for care that have already been performed. Such tasks are marked or recorded as performed. Any tasks that should have been performed are scheduled with a greater priority in order to maintain the timeline for the updated workflow of care.

By reestablishing the workflow for care, the patient may be assigned to a more current or accurate severity or diagnosis-related group. A more optimized or appropriate workflow for care is performed. If performed in real time, suggestions and corrections can be made to improve the quality of care. For example, from the present symptoms, lab and imaging results, suggestions and communications can be made to elevate the severity of a patient from acute renal insufficiency to acute renal failure. This may not only yield a better outcome if confirmed but also may save time and resources.

The update occurs while the patient is at the healthcare facility, maximizing or increasing the opportunity to provide efficient and appropriate care. The update may result in cost savings, increased reimbursement opportunity, and/or more optimum care. The update may avoid increasing costs or reducing care due to performing actions not needed given the diagnosis-related group to which the patient now belongs.

The classification update is made during the patient stay. The classification may be repeated at different times during the patient stay. The classification is updated, such updated based on any data entered after the original classification.

In act 410, tasks are scheduled based on the timeline. The tasks are scheduled automatically. The system populates the calendars or task lists of different personnel, equipment, rooms, or other resources. For example, a time for medical imaging equipment and room is reserved, and the calendar of a technician for the medical imaging system is changed to indicate an appointment for that time. Any task to be performed by someone or something is a job entry. Reservations may be scheduled in addition to or as a job entry. Tasks may be added to the workflows of different people.

In another embodiment, a job entry in a workflow of care is automatically scheduled. The computerized workflow system includes action items to be performed by different individuals. The action items are communicated to the individual in a user interface for the workflow, by email, by text message, by placement in a calendar, or by other mechanism.

The automated scheduling may be subject to approval by one or more people. The technician, physician, or nurse may be required to accept any scheduled appointment. Where an appointment is rejected, the timeline may be adjusted to a next optimal time. In another example of approval, a case manager may be required to approve of the entire timeline and/or any changes to the timeline before scheduling is attempted and/or completed.

In one embodiment, a job entry is added to the workflow of a case manager. In a retrospective analysis or in real time after identification of a problem or issue, the case manager may be tasked with avoiding the problem or issue for the same patient or other patients with a same or similar workflow of care. For example, a patient or threshold number of patients is readmitted to a hospital due to a complication. The case manager may be tasked with attempting to prevent readmission of other patients with the same workflow of care. To avoid readmission, the case manager identifies cost effective actions, such as education about post discharge treatment. The actions are added to the workflow for care as an update. The case management workflow system may monitor for issues and generate tasks or suggest changes to deal with the issues.

In act 412, the processor generates at least one alert. The system may be configured to monitor adherence to the action items of the workflow for care. Reminders may be automatically generated where an action item is due or past due so that health care providers are better able to follow the timeline.

The timeline provides a schedule. Alerts are generated for conflicts with the schedule, such as physician being double booked. Alerts are generated as reminders for an upcoming action. Alerts are generated for administrators, nurses or others to cause another person to act on time. Alerts are generated where an action should have occurred and data entered, but where data has not been entered. Alerts may be generated for any reason in an effort to keep to the timeline or limit further delay than has already happened.

Any type of alert may be used. The alert is sent via text, email, voice mail, voice response, or network notification. The alert indicates the task to be performed, the location, and the patient. The alert is sent to the patient, family member, treating physician, nurse, primary care physician, and/or other medical professional. The alert may be transmitted to a computer, cellular phone, tablet, bedside monitor of the patient, or other device. The alert may be communicated through a workflow system. For example, a task to be performed is highlighted when past due or due soon. The highlighting may indicate a cost for selecting between multiple past or currently due tasks. The alert may be sent to the workflow of others for analysis, such as to identify people that regularly fail to perform on time so that future costs may be saved through training or education.

The alert may include additional information. The alert may indicate a cost associated with failure to perform on time. The diagnosis grouping, recently acquired data, relevant data, the severity, a probability associated with treatment, treatment options, or other information may be included.

In one embodiment, the alert is generated as a displayed warning while preventing entry of other information. The user is prevented from some action, task, or data entry to require submission of documentation of the act or other acts. For example, in response to the user attempting to schedule discharge or enter information associated with the patient, the alert is generated and the user is prevented from entering or saving the information. The prevention is temporary (e.g., seconds or minutes), may remain until the missing information is provided, or require an over-ride from an authorized personnel (e.g. a case manager or an attending physician). The prevention may be for one type of data entry (e.g., discharge scheduling) but allow another type (e.g., medication reconciliation) to reduce the risk of costly extensions.

In act 414, the processor predicts a probability of meeting the timeline, a cost associated with meeting the timeline, and a strongest link to the probability indicating a risk of failure to meet the timeline. Additional, different, or fewer items may be predicted. The prediction is based on past performance or a study. For example, the rate of timeline compliance is measured as performing every action on time, discharging on time or other measure. Previous implementations of a given workflow of care may be measured. The rate of compliance provides a probability of meeting the timeline. The probability is by physician, facility, practice group, or general. In alternative embodiments, the probability may be predicted by a machine-learned classifier based on training data of pervious patients.

The cost is predicted based on study or domain knowledge. For example, costs associated with performing the workflow of care over different timelines may be determined. The cost may be in terms of financial cost, resource utilization, reimbursement or difference between reimbursement and cost to perform the workflow. Based on financial study, the cost information may indicate the financial result of delay. Incentives and/or penalties may be associated with failure to perform on time. The costs may be broken down into components, such as the cost associated with each action or task.

The strongest link to the probability indicating a risk of failure to meet the timeline may be provided to a case manager or person associated with the strongest link. By providing the link, tasks may be handled more efficiently and/or to more likely avoid delay. The strongest link may be the most frequent cause for delay as compared to various causes of delay (e.g., people or equipment) or another less frequent cause associated with greater cost. The risk for delay may be linked to one of various variables. The variable with the strongest link is the most frequent cause, the cause of the longest delays, or cause associated with greater costs relative to other variables.

The probability, cost, or link may be specific to a hospital, physician, practice group or other entity, such as being calculated based on data for the hospital. Alternatively, the probability, cost, or link is based on peer performance or is general.

The implementation of the computerized case management may be based on a criteria set for the medical entity. For example, the medical entity may set the threshold for comparison to be more or less inclusive of different levels of performance or cost. As another example, the medical entity may select a combination of factors to trigger an alert. If one variable causes the case management system to regularly and inaccurately predict a class, then some values of that variable may cause an alert to be generated for a case manager to more completely review the established workflow of care.

The workflow of care for the patient is for the patient's stay at the healthcare facility. The same workflow or a further, different workflow may be established for the care of the patient after a discharge.

FIG. 2 shows one embodiment of a method for computer-based patient management for healthcare. The method is directed to accountable care optimization. In addition to or as an alternative to case management for patients at a healthcare facility, the patients are managed prior to and/or after any stay. By managing possible patients outside of the healthcare facility, the overall cost of healthcare may be reduced. The care optimization workflow is performed, at least in part, by a computer or automatically.

The acts are performed in the order shown or a different order. For example, act 508 is performed as part of act 504. Additional, different, or fewer acts may be performed. For example, one or two of acts 504, 506, and 508 are not performed. Acts 510, 512, 514, and 516 are performed in parallel, not provided, or are alternatives.

In act 502, clinical data is gathered. A processor acquires the data. Data for a patient is acquired by searching the medical record or other information sources for the patient. In one embodiment, mining or other gathering discussed for act 402 and/or or 406 is performed. For example, unstructured information is mined. The mining provides values for variables where the values are inferred from different possible values and probabilities assigned to the possible values.

In act 504, the processor establishes care for a patient not at a healthcare facility. The workflow for care is established prior to a given admission to a healthcare facility. For example, the workflow for care is an ongoing process established by an insurance company, medical facility, primary care physician, or other group to prevent injury or illness. The goal is to avoid any hospital admissions or more costly procedures. Thus, the workflow for care is established regardless of whether there will be a later admission (prior to any later admission). Alternatively, the workflow for care is established after an admission is planned but prior to the actual admission.

The care for the patient is established as a function of the data for the patient. A cohort for the patient is predicted. A classifier, such as a classifier discussed above for act 404, determines a cohort to which a patient belongs. The classification may be based on a diagnosis or not. For example, cohorts are groups of the population with similar health concerns or risks. Whether a patient has been vaccinated or not, the weight of the patient, allergies, which allergies, diabetes, and/or other information may be used to group patients into different cohorts. Each cohort is associated with different types of risk, levels or severity of risk, and/or combinations of risks.

The available workflows for care may define the possible cohorts. Different combinations of concerns may lead to different care. The care is provided to manage risk and avoid more expensive health complications. By establishing care prior to any more major illnesses or injuries, actions may be taken to reduce costs for later care. The care may be provided as part of accountable care optimization, such as attempting to reduce costs of healthcare by managing the person rather than case managing after injury or illness has occurred.

As new data is acquired, the care may be updated, such as disclosed in act 408 but for treatment or care outside of the healthcare facility. For example, a patient may suffer a fall or a plurality of falls within a given time frame. Such falls may be identified by data indicating calls to a healthcare provider, incident reports at a senior living facility or other sources. This new data may be used to reassign the patient to a different cohort, resulting in different care processes. A personalized plan of care is provided for each patient. Patient is used for people that may or may not be a patient of a physician, but are patients in the sense of people for which care is to be provided.

The workflow of care outside of the healthcare facility may include different actions. The actions may be for the patient to perform, such as membership at a health club or visiting a physician. The actions may be for others to perform, such as monitoring, home visits, calls, other contact, or other interaction to encourage, require, or test the patient. The care may be monitored by requiring entry of feedback by the patient and/or by acquiring data associated with the care.

The system may automatically monitor or schedule the care. The monitoring or scheduling may be in a timeline or otherwise arranged to minimize costs. For example, statistics may be used to determine the most cost effective approaches. Where visits to nurse practitioners are successful, such visits are arranged for a given task. Where such visits frequently lead to physician visits, the nurse practitioner approach is not used first.

In act 506, the care for the patient upon admission to a healthcare facility is managed. The management may be automated, such as discussed above for FIG. 1, may be managed by a case manager, or combinations thereof. The management is performed using data for the patient.

In act 508, the processor establishes care for the patient after any discharge from the healthcare facility. The care is established as discussed above for act 504, 404, or 408. The classifier is the same or different for each of these acts. For example, a general classifier appropriate for acts 504 and 508 is used. In another example, separate classifiers are trained or provided for acts 504 and 508 given the different circumstances.

Currently available data is used to classify the patient into a cohort for assigning a care plan to the patient. The data includes data associated with the treatment at the healthcare facility, so may classify the patient into a cohort associated with care appropriate for the patient given the diagnosis.

For example, an optimal follow-up strategy (e.g., phone call, in-home follow-up, or visit to a doctor) may be provided in the care plan. The follow-up strategy may be selected or determined based on the probability of readmission, probability of compliance, guidelines for care, and/or the variables associated with the patient. For example, an in-home follow-up is scheduled for a probability of compliance further beyond (e.g., below) the threshold (e.g., beyond another threshold in a stratification of risk), and a phone call is scheduled for a probability closer to the threshold (e.g., for a lower risk). As another example, the severity or cost of the illness, injury, or health risk is used to select the appropriate care. Possible and alternative care plans for optimal patient outcomes may be provided for selection with or without cost considerations.

Other predictors or statistical classifiers may be provided. One example predictor is for compliance by the patient with instructions. A level of risk (i.e., risk stratification) and/or reasons for risk are predicted. The ground truth for compliance may rely on patient surveys or questionnaires. The predictor for whether a patient will comply is trained from training data. Different predictors may be generated for different groups, such as by type of condition, cohort, or diagnosis-related group. The variables used for training may be the same or different than for training a predictor of timeline performance. Mining is performed to determine the values for training and/or the values for application.

The predictor for compliance is triggered for application at the time of discharge or when other instructions are given to the patient, but may be performed at other times. The values of variables in the feature vector of the predictor of compliance are input to the predictor. The application of the predictor to the electronic medical record of the patient results in an output probability of compliance by the patient. The reasons for the probability being beyond a threshold or thresholds may also be output, such as a lack of insurance or high medication cost contributing as a strong or stronger link to the probability being beyond the threshold. For example, a patient may be discharged to an unknown location (no home or hospice listed in the discharge location variable). An unknown location may occur for homeless patients whom are less able to adhere to a care plan. The discharge location being unknown may be output so that a care provider may make subsequent care arrangements before discharge or assign a case worker to assist with adherence. Alternatively, the management workflow identifies the situation and arranged for assignment of the case work, visit by a case worker, or return visits to a healthcare facility.

The probability of compliance may be used to modify the discharge or other instructions and/or workflow action items. For example, the type of follow-up may be more intensive or thorough where the probability of compliance is low. As another example, a workflow action may be generated to identify alternative medicines where the cost of medication is high. A consultation with a social worker may be arranged and/or the discharge instructions based on lower cost alternatives may be provided where the patient does not have insurance. The timeline for care may be altered to provide for further tasks associated with compliance or reduction of other risks.

The probability of readmission may be predicted, such as disclosed in U.S. Published Application No. _______________ (Ser. No. 13/153,551), the disclosure of which is incorporated herein by reference. The probability of readmission may be used to identify care to avoid readmission. A risk of readmission is predicted and care to mitigate the risk may be provided in the workflow for care of the patient.

Other predictions may be used, such as predicting a financial impact and/or predicting a severity. Probabilities or other predictive information may be used to establish the care for the patient. Different tasks may be assigned as a function of the severity and/or financial impact. More severe or costly risks may result in a care plan with more intensive care and corresponding tasks to avoid the risk.

Any of the care plans of acts 504, 506, and 508 may be updated. As new data is acquired, the cohort assignment may be updated, resulting in a different care plan. Different cohorts may have different plans.

The care plans may be established, at least in part, based on utilization. Different actions are associated with different costs. Less expensive alternatives may be used where care does not suffer. For example, actions associated with less resource consumption are used while still satisfying treatment guidelines. The cost may be accounted for in any of the ways discussed above for FIG. 1 even in managing patients outside of healthcare facilities.

In act 510, tasks for the care are generated or scheduled by the processor. To reduce, minimize or avoid case manager workload, the management of the care is performed automatically. The reminders, tasks, scheduling, or actions are assigned and monitored. For example, visits to healthcare providers are scheduled. A notice may be sent to the patient, allowing the patient to interact with the schedule of the physician to arrange for a visit. Exercise, support group, or other activities may be scheduled or arranged. Any of the tasks for the care may be altered due to new data, case manager override, or other circumstances. The system reacts to changes by attempting to satisfy the care plan as currently provided.

The management of the care occurs across different medical providers associated with different institutions. The system may interact with different formats or systems at the different entities.

In act 512, the processor generates an alert, reminder, or task as part of the care before, during, and/or after admission to a healthcare facility. The alerts, reminders, or tasks are handled in the same way as provided in act 412. For care outside the healthcare facility, the alerts may more likely be to the patient or family member of the patient. The alerts may make compliance more likely. The alerts or reminders may be provided to a case manager, such as to arrange for a call or face-to-face consultation to increase the rate of compliance.

In act 514, information associated with the care before, during, or after a stay at a healthcare facility is presented to a user, such as a case manager, nurse, physician, administrator, patient, or other party. The information presented may include the care plan, the schedule or timeline, or other information. For example, the information indicates completed and incomplete tasks. Probabilities of compliance, of meeting the care plan, of readmission, or of other events may be presented. The probabilities may be provided with possible modifications to the care plan or indication of variables most likely to mitigate risk.

The information presented as part of the case management system may be different for different people. People with different roles receive access to different information. For example, a physician may access information on scheduled care tasks, but not financial information. A patient may receive information on the care plan, but not reminders to others. A case manager may receive access to the entire care plan, including financial information.

In act 516, the processor determines and indicates performance information. The performance information may be used by the case management system or a case manager to provide more effective care and/or more cost effective care. Physicians with patients that more likely comply or avoid admissions may be utilized more than other physicians. Healthcare facilities using less costly procedures or resources with similar success or care may be used over other facilities.

The performance is calculated based on data. Any criteria may be used for measurement. The data from past patients for a given physician, healthcare facility, or other entity is obtained and used to determine statistics. For example, the rates of vaccination of patients by different physicians are determined. Since vaccination may avoid later costs, the cost benefit associated with this statistic or the statistic itself is used to control the management workflow. The computer attempts to schedule visits with the physicians with a greater rate of vaccination first.

The performance may be indicated to a case manager for review. Workflows or limitations of operation of the management system may be altered to account for performance.

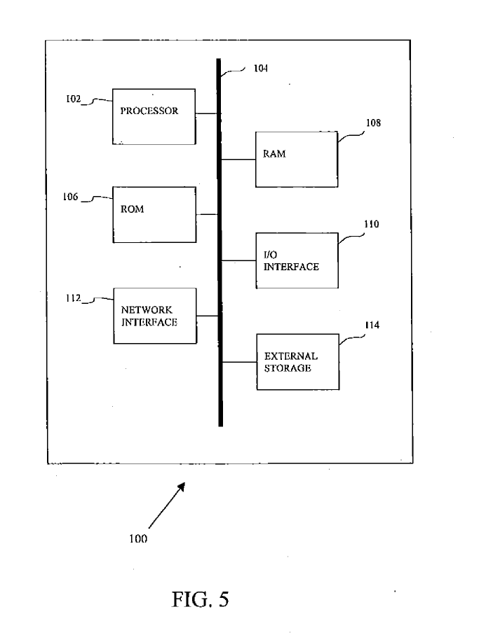

FIG. 5 is a block diagram of an example computer processing system 100 for implementing the embodiments described herein, such as computer-based patient management for healthcare. The systems, methods and/or computer readable media may be implemented in various forms of hardware, software, firmware, special purpose processors, or a combination thereof. Some embodiments are implemented in software as a program tangibly embodied on a program storage device. By implementing with a system or program, completely or semi-automated workflows, predictions, classifying, and/or data mining are provided to assist a person or medical professional.

The system 100 may be for generating a classifier, such as implementing machine learning to train a statistical classifier. Alternatively or additionally, the system 100 is for applying the classifier. The system 100 may also or alternatively implement associated workflows.

The system 100 is a computer, personal computer, server, PACs workstation, imaging system, medical system, network processor, or other now know or later developed processing system. The system 100 includes at least one processor (hereinafter processor) 102 operatively coupled to other components via a system bus 104. The program may be uploaded to, and executed by, a processor 102 comprising any suitable architecture. Likewise, processing strategies may include multiprocessing, multitasking, parallel processing and the like. The processor 102 is implemented on a computer platform having hardware such as one or more central processing units (CPU), a random access memory (RAM), and input/output (I/O) interface(s). The computer platform also includes an operating system and microinstruction code. The various processes and functions described herein may be either part of the microinstruction code or part of the program (or combination thereof) which is executed via the operating system. Alternatively, the processor 102 is one or more processors in a network and/or on an imaging system.

The processor 102 is configured to learn a classifier, such as creating a classifier for severity, diagnosis-related grouping, cohort grouping, predicting (e.g., compliance, readmission, ability to meet a timeline or other event), or clustering from training data, to mine the electronic medical record of the patient or patients, and/or to apply a machine-learnt classifier to implement case management or accountable care optimization. Training and application of a trained classifier are first discussed below. Example embodiments for mining follow.

For training, the processor 102 determines the relative or statistical contribution of different variables to the outcome—severity, diagnosis-related group, or cohort. A programmer may select variables to be considered. The programmer may influence the training, such as assigning limitations on the number of variables and/or requiring inclusion of one or more variables to be used as the input feature vector of the final classifier. By training, the classifier identifies variables contributing to establishing a workflow. Where the training data is for patients from a given medical entity, the learning identifies the variables most appropriate or determinative for that medical entity. The training incorporates the variables into a classifier for a future patient of the medical entity. For example, the training provides a classifier to output one or more diagnoses for a given patient so that the diagnoses may be used to select, in combination, the appropriate care workflow.

For application, the processor 102 applies the resulting (machine-learned) statistical model to the data for a patient. For each patient or for each patient in a category of patients (e.g., patients treated for a specific condition or by a specific group within a medical entity), the classifier is applied to the data for the patient. The values for the identified and incorporated variables of the machine-learnt statistical model are input as a feature vector. A matrix of weights and combinations of weighted values calculates a class, diagnosis-related group, or cohort.

The processor 102 associates different workflows of care with different possible classes of the classifier. The diagnosis-related grouping, cohort, probability, severity, and/or most determinative values may be different for different patients. One or a combination of these factors is used to select an appropriate workflow or action. Different classes may result in different jobs to be performed, different timelines and/or different cost considerations.

The processor 102 is operable to assign actions or to perform management workflow actions. For example, the processor 102 initiates contact for follow-up by electronically notifying a patient in response to identifying a care plan. As another example, the processor 102 requests documentation to resolve ambiguities in a medical record. In another example, the processor 102 generates a request for clinical action likely to provide better care and/or utilization. Clinical actions may include a test, treatment, visit, other source of obtaining clinical information, or combinations thereof. To implement case management, the processor 102 may generate a prescription form, clinical order (e.g., test order), treatment, visit, appointment, activity, or other workflow action.

In a real-time usage, the processor 102 receives currently available medical information for a patient. Based on the currently available information and mining the patient record, the processor 102 may indicate a currently appropriate class and/or establish a patient-appropriate workflow of care. The actions may then be performed during the treatment or before discharge. The processor 102 may arrange for actions to occur outside of a healthcare facility.

In one embodiment represented in FIG. 6, a database 310 storing a patient record is mined by a miner 350 implemented by a processor to output structured data 380. A same or different processor uses the structured data, such as in an input feature vector of a machine-learned classifier, as values for variables used to establish a workflow for care, or to implement the workflow for care as a case management system. For example, a severity 382 is predicted. The diagnosis-related group 384 is predicted from the structured data and/or is derived from the severity 382. Each of the patients is classified into diagnosis-related groups based on respective data for each of the patients, whether indirectly through classification of severity or directly by classification into the group. A care plan 388 and a timeline 386 are identified based on the diagnosis-related group 384 and/or the severity 382. For case management at a healthcare facility, the timeline 386 is to discharge and is selected as a function of the diagnosis-related group for each of the patients. The processor may then implement the timeline 386 and care plan 388.

The diagnosis-related group may be altered for at least one of the patients. The altering is based on a utilization and new data not previously used in the classifying. The utilization may be realized by considering a cost factor in the creation of workflows, in the classification of the patient, and/or in actions selected in the timeline. The diagnosis-related group for the one or more patients is changed. The changing is a function of cost of the tasks, reimbursement for the tasks, and/or the new data. The new data may be obtained after the classifying and while the patient is being treated at a healthcare facility. Due to the change, the timeline 386 for the patient may change.

The processor monitors tasks across multiple medical professionals as a function of the timeline. To manage the care, an alert is generated in response to the monitoring, such as to more effectively implement the timeline.

Referring again to FIG. 5, the processor 102 implements the operations as part of the system 100 or a plurality of systems. A read-only memory (ROM) 106, a random access memory (RAM) 108, an I/O interface 110, a network interface 112, and external storage 114 are operatively coupled to the system bus 104 with the processor 102. Various peripheral devices such as, for example, a display device, a disk storage device(e.g., a magnetic or optical disk storage device), a keyboard, printing device, and a mouse, may be operatively coupled to the system bus 104 by the I/O interface 110 or the network interface 112.

The computer system 100 may be a standalone system or be linked to a network via the network interface 112. The network interface 112 may be a hard-wired interface. However, in various exemplary embodiments, the network interface 112 may include any device suitable to transmit information to and from another device, such as a universal asynchronous receiver/transmitter (UART), a parallel digital interface, a software interface or any combination of known or later developed software and hardware. The network interface may be linked to various types of networks, including a local area network (LAN), a wide area network (WAN), an intranet, a virtual private network (VPN), and the Internet.

The instructions and/or patient record are stored in a non- transitory computer readable memory, such as the external storage 114. The same or different computer readable media may be used for the instructions and the patient record data. The external storage 114 may be implemented using a database management system (DBMS) managed by the processor 102 and residing on a memory such as a hard disk, RAM, or removable media. Alternatively, the storage 114 is internal to the processor 102 (e.g. cache). The external storage 114 may be implemented on one or more additional computer systems. For example, the external storage 114 may include a data warehouse system residing on a separate computer system, a PACS system, or any other now known or later developed hospital, medical institution, medical office, testing facility, pharmacy or other medical patient record storage system. The external storage 114, an internal storage, other computer readable media, or combinations thereof store data for at least one patient record for a patient. The patient record data may be distributed among multiple storage devices or in one location.

The patient data for training a machine learning classifier is stored. The training data includes data for patients that have been classified, such as by a case manager or physician. The training data may additionally or alternatively include records of timeline implementation. The patients are for a same medical entity, group of medical entities, region, or other collection.

Alternatively or additionally, the data for applying a machine-learnt classifier is stored. The data is for a patient being managed. The memory stores the electronic medical record of one or more patients. Links to different data sources may be provided or the memory is made up of the different data sources. Alternatively, the memory stores extracted values for specific variables.

The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system. Because some of the constituent system components and method steps depicted in the accompanying figures are preferably implemented in software, the actual connections between the system components (or the process steps) may differ depending upon the manner in which the present embodiments are programmed.

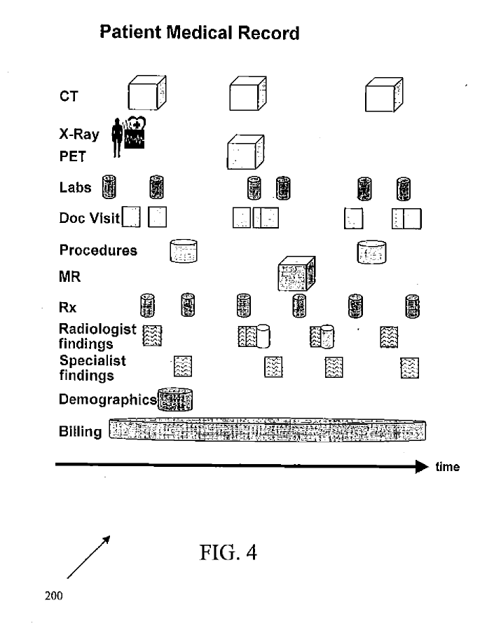

Healthcare providers may employ automated techniques for information storage and retrieval. The use of a computerized patient record (CPR)(e.g., an electronic medical record) to maintain patient information is one such example. As shown in FIG. 4, an exemplary CPR 200 includes information collected over the course of a patient's treatment or use of an institution. This information may include, for example, computed tomography (CT) images, X-ray images, laboratory test results, doctor progress notes, details about medical procedures, prescription drug information, radiological reports, other specialist reports, demographic information, family history, patient information, and billing (financial) information.

A CPR may include a plurality of data sources, each of which typically reflects a different aspect of a patient's care. Alternatively, the CPR is integrated into one data source. Structured data sources, such as financial, laboratory, and pharmacy databases, generally maintain patient information in database tables. Information may also be stored in unstructured data sources, such as, for example, free text, images, and waveforms. Often, key clinical findings are only stored within unstructured physician reports, annotations on images or other unstructured data source.

Referring to FIG. 2, the processor 102 executes the instructions stored in the computer readable media, such as the storage 114. The instructions are for mining patient records (e.g., the CPR), computer-based patient management for healthcare, predicting readmission, assigning workflow jobs, other functions, or combinations thereof. For training and/or application of the classifier or management of care, values of variables are used. The values for particular patients are mined from the CPR. The processor 102 mines the data to provide values for the variables.

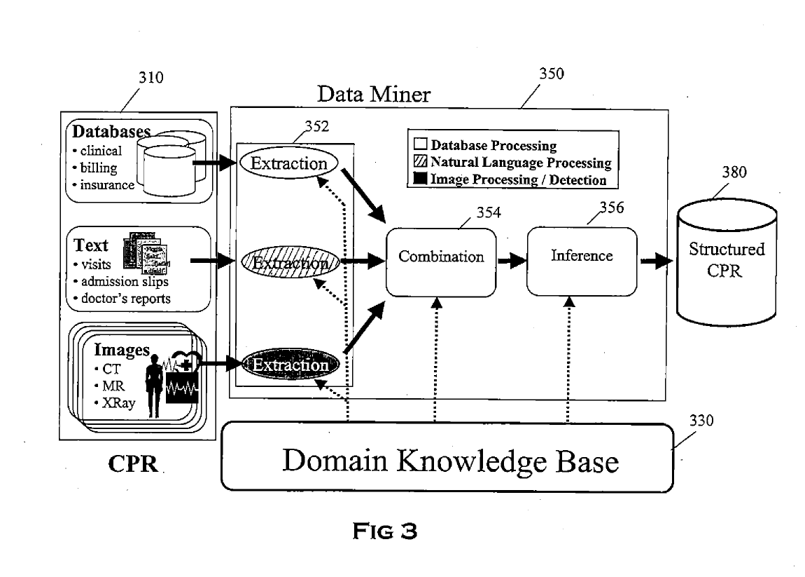

Any technique may be used for mining the patient record, such as structured data based searching. In one embodiment, the methods, systems and/or instructions disclosed in U.S. Published Application No. 2003/0120458 are used, such as for mining from structured and unstructured patient records. FIG. 3 illustrates an exemplary data mining system implemented by the processor 102 for mining a patient record to create high-quality structured clinical information. The processing components of the data mining system are software, firmware, microcode, hardware, combinations thereof, or other processor based objects. The data mining system includes a data miner 350 that mines information from a CPR 310 using domain-specific knowledge contained in a knowledge base 330. The data miner 350 includes components for extracting information from the CPR 352, combining all available evidence in a principled fashion over time 354, and drawing inferences from this combination process 356. The mined information may be stored in a structured CPR 380. The architecture depicted in FIG. 4 supports plug-in modules wherein the system may be easily expanded for new data sources, diseases, and hospitals. New element extraction algorithms, element combining algorithms, and inference algorithms can be used to augment or replace existing algorithms.

The mining is performed as a function of domain knowledge. The domain knowledge provides an indication of reliability of a value based on the source or context. For example, a note indicating the patient is a smoker may be accurate 90% of the time, so a 90% probability is assigned. A blood test showing nicotine may indicate that the patient is a smoker with 60% accuracy.

Detailed knowledge regarding the domain of interest, such as, for example, a disease of interest, guides the process to identify relevant information. This domain knowledge base 330 can come in two forms. It can be encoded as an input to the system, or as programs that produce information that can be understood by the system. For example, a study determines factors contributing to timeline completion or class assignment. These factors and their relationships may be used to mine for values. The study is used as domain knowledge for the mining. Additionally or alternatively, the domain knowledge base 330 may be learned from test data.

The domain-specific knowledge may also include disease- specific domain knowledge. For example, the disease-specific domain knowledge may include various factors that influence risk of a disease, disease progression information, complications information, outcomes, and variables related to a disease, measurements related to a disease, and policies and guidelines established by medical bodies.

The information identified as relevant by the study, guidelines for treatment, medical ontologies, or other sources provides an indication of probability that a factor or item of information indicates or does not indicate a particular value of a variable. The relevance may be estimated in general, such as providing a relevance for any item of information more likely to indicate a value as 75% or other probability above 50%. The relevance may be more specific, such as assigning a probability of the item of information indicating a particular diagnosis based on clinical experience, tests, studies or machine learning. Based on the domain-knowledge, the mining is performed as a function of existing knowledge, guidelines, or best practices regarding care. The domain knowledge indicates elements with a probability greater than a threshold value of indicating the patient state (i.e., collection of values). Other probabilities may be associated with combinations of information.

Domain-specific knowledge for mining the data sources may include institution-specific domain knowledge. For example, information about the data available at a particular hospital, document structures at a hospital, policies of a hospital, guidelines of a hospital, and any variations of a hospital. The domain knowledge guides the mining, but may guide without indicating a particular item of information from a patient record.

The extraction component 352 deals with gleaning small pieces of information from each data source regarding a patient or plurality of patients. The pieces of information or elements are represented as probabilistic assertions about the patient at a particular time. Alternatively, the elements are not associated with any probability. The extraction component 352 takes information from the CPR 310 to produce probabilistic assertions (elements) about the patient that are relevant to an instant in time or period. This process is carried out with the guidance of the domain knowledge that is contained in the domain knowledge base 330. The domain knowledge for extraction is generally specific to each source, but may be generalized.

The data sources include structured and/or unstructured information. Structured information may be converted into standardized units, where appropriate. Unstructured information may include ASCII text strings, image information in DICOM (Digital Imaging and Communication in Medicine) format, and text documents partitioned based on domain knowledge. Information that is likely to be incorrect or missing may be noted, so that action may be taken. For example, the mined information may include corrected information, including corrected ICD-9 diagnosis codes.

Extraction from a database source may be carried out by querying a table in the source, in which case, the domain knowledge encodes what information is present in which fields in the database. On the other hand, the extraction process may involve computing a complicated function of the information contained in the database, in which case, the domain knowledge may be provided in the form of a program that performs this computation whose output may be fed to the rest of the system.

Extraction from images, waveforms, etc., may be carried out by image processing or feature extraction programs that are provided to the system.

Extraction from a text source may be carried out by phrase spotting, which requires a list of rules that specify the phrases of interest and the inferences that can be drawn there from. For example, if there is a statement in a doctor's note with the words “There is evidence of metastatic cancer in the liver,” then, in order to infer from this sentence that the patient has cancer, a rule is needed that directs the system to look for the phrase “metastatic cancer,” and, if it is found, to assert that the patient has cancer with a high degree of confidence (which, in the present embodiment, translates to generate an element with name “Cancer”, value “True” and confidence 0.9).

The combination component 354 combines all the elements that refer to the same variable at the same time period to form one unified probabilistic assertion regarding that variable. Combination includes the process of producing a unified view of each variable at a given point in time from potentially conflicting assertions from the same/different sources. These unified probabilistic assertions are called factoids. The factoid is inferred from one or more elements. Where the different elements indicate different factoids or values for a factoid, the factoid with a sufficient (thresholded) or highest probability from the probabilistic assertions is selected. The domain knowledge base may indicate the particular elements used. Alternatively, only elements with sufficient determinative probability are used. The elements with a probability greater than a threshold of indicating a patient state (e.g., directly or indirectly as a factoid), are selected. In various embodiments, the combination is performed using domain knowledge regarding the statistics of the variables represented by the elements (“prior probabilities”).

The patient state is an individual model of the state of a patient. The patient state is a collection of variables that one may care about relating to the patient, such as established by the domain knowledgebase. The information of interest may include a state sequence, i.e., the value of the patient state at different points in time during the patient's treatment.

The inference component 356 deals with the combination of these factoids, at the same point in time and/or at different points in time, to produce a coherent and concise picture of the progression of the patient's state over time. This progression of the patient's state is called a state sequence. The patient state is inferred from the factoids or elements. The patient state or states with a sufficient (thresholded), high probability or highest probability is selected as an inferred patient state or differential states.

Inference is the process of taking all the factoids and/or elements that are available about a patient and producing a composite view of the patient's progress through disease states, treatment protocols, laboratory tests, clinical action or combinations thereof. Essentially, a patient's current state can be influenced by a previous state and any new composite observations. The diagnosis-related group, severity, cohort, or other item to be predicted, classified or clustered may be considered as a patient state so that the mining determines the item without a further application of a separate model.

The domain knowledge required for this process may be a statistical model that describes the general pattern of the evolution of the disease of interest across the entire patient population and the relationships between the patient's disease and the variables that may be observed (lab test results, doctor's notes, or other information). A summary of the patient may be produced that is believed to be the most consistent with the information contained in the factoids, and the domain knowledge.

For instance, if observations seem to state that a cancer patient is receiving chemotherapy while he or she does not have cancerous growth, whereas the domain knowledge states that chemotherapy is given only when the patient has cancer, then the system may decide either: (1) the patient does not have cancer and is not receiving chemotherapy (that is, the observation is probably incorrect), or (2) the patient has cancer and is receiving chemotherapy (the initial inference—that the patient does not have cancer—is incorrect); depending on which of these propositions is more likely given all the other information. Actually, both (1) and (2) may be concluded, but with different probabilities.

As another example, consider the situation where a statement such as “The patient has metastatic cancer” is found in a doctor's note, and it is concluded from that statement that <cancer=True (probability=0.9)>. (Note that this is equivalent to asserting that <cancer=True (probability=0.9), cancer=unknown (probability=0.1)>).

Now, further assume that there is a base probability of cancer <cancer=True (probability=0.35), cancer=False (probability=0.65)>(e.g., 35% of patients have cancer). Then, this assertion is combined with the base probability of cancer to obtain, for example, the assertion <cancer=True (probability=0.93), cancer=False (probability=0.07)>.

Similarly, assume conflicting evidence indicated the following:
1. <cancer=True (probability=0.9), cancer=unknown probability=0.1)>
2. <cancer=False (probability=0.7), cancer=unknown (probability=0.3)>
3. <cancer=True (probability=0.1), cancer=unknown (probability=0.9)> and
4. <cancer=False (probability=0.4), cancer=unknown (probability=0.6)>.

In this case, we might combine these elements with the base probability of cancer <cancer=True (probability=0.35), cancer=False (probability=0.65)> to conclude, for example, that <cancer=True (prob =0.67), cancer=False (prob=0.33)>.

Numerous data sources may be assessed to gather the elements, and deal with missing, incorrect, and/or inconsistent information. As an example, consider that, in determining whether a patient has diabetes, the following information might be extracted:
(a) ICD-9 billing codes for secondary diagnoses associated with diabetes;
(b) drugs administered to the patient that are associated with the treatment of diabetes (e.g., insulin);
(c) patient's lab values that are diagnostic of diabetes (e.g., two successive blood sugar readings over 250 mg/d);
(d) doctor mentions that the patient is a diabetic in the H&P (history & physical) or discharge note (free text); and
(e) patient procedures (e.g., foot exam) associated with being a diabetic.
   As can be seen, there are multiple independent sources of information, observations from which can support (with varying degrees of certainty) that the patient is diabetic (or more generally has some disease/condition). Not all of them may be present, and in fact, in some cases, they may contradict each other. Probabilistic observations can be derived, with varying degrees of confidence. Then these observations (e.g., about the billing codes, the drugs, the lab tests, etc.) may be probabilistically combined to come up with a final probability of diabetes. Note that there may be information in the patient record that contradicts diabetes. For instance, the patient has some stressful episode (e.g., an operation) and his blood sugar does not go up.

The above examples are presented for illustrative purposes only and are not meant to be limiting. The actual manner in which elements are combined depends on the particular domain under consideration as well as the needs of the users of the system. Further, while the above discussion refers to a patient-centered approach, actual implementations may be extended to handle multiple patients simultaneously. Additionally, a learning process may be incorporated into the domain knowledge base 330 for any or all of the stages (i.e., extraction, combination, inference).

The system may be run at arbitrary intervals, periodic intervals, or in online mode. When run at intervals, the data sources are mined when the system is run. In online mode, the data sources may be continuously mined. The data miner may be run using the Internet. The created structured clinical information may also be accessed using the Internet. Additionally, the data miner may be run as a service. For example, several hospitals may participate in the service to have their patient information mined, and this information may be stored in a data warehouse owned by the service provider. The service may be performed by a third party service provider (i.e., an entity not associated with the hospitals).

Once the structured CPR 380 is populated with patient information, it will be in a form where it is conducive for answering questions regarding individual patients, and about different cross-sections of patients. The values are available for use in classifying the patient to determine a workflow for care.

The domain knowledgebase, extractions, combinations and/or inference may be responsive or performed as a function of one or more variables. For example, the probabilistic assertions may ordinarily be associated with an average or mean value. However, some medical practitioners or institutions may desire that a particular element be more or less indicative of a patient state. A different probability may be associated with an element. As another example, the group of elements included in the domain knowledge base for a case manager may be different for different medical entities. The threshold for sufficiency of probability or other thresholds may be different for different people or situations.

Other variables may be use or institution specific. For example, different definitions of a primary care physician may be provided. A number of visits threshold may be used, such as visiting the same doctor 5 times indicating a primary care physician. A proximity to a patient's residence may be used. Combinations of factors may be used.

The user may select different settings. Different users in a same institution or different institutions may use different settings. The same software or program operates differently based on receiving user input. The input may be a selection of a specific setting or may be selection of a category associated with a group of settings.

The mining, such as the extraction, and/or the inferring, such as the combination, are performed as a function of the selected threshold. By using a different upper limit of normal for the patient state, a different definition of information used in the domain knowledge or other threshold selection, the patient state or associated probability may be different. User's with different goals or standards may use the same program, but with the versatility to more likely fulfill the goals or standards.

Various improvements described herein may be used together or separately. Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

What is claimed is:

1. A method for computer-based patient management for healthcare, the method comprising:
gathering, with a processor, first clinical data for a patient of a healthcare facility;
establishing, with the processor, a workflow for care of the patient as a function of the first clinical data and a cost factor, the workflow being for multiple actions by different entities of the healthcare facility and including a timeline for the actions;
gathering, with the processor, second clinical data after establishing and as part of the workflow for the care of the patient;
updating, with the processor, the workflow for the care of the patient as a function of the first and second clinical data and the cost factor, the updating occurring while the patient is at the healthcare facility; and
generating, with the processor, at least one alert for at least one of the multiple actions, the alert generated as a function of the timeline.

2. The method of claim 1 wherein establishing the workflow comprises predicting a severity and assigning the patient to a diagnosis-related group as a function of the severity.

3. The method of claim 1 wherein gathering comprises mining an electronic medical record of the patient, and wherein establishing comprise populating a feature vector with the mining and predicting a probability from the feature vector.

4. The method of claim 3 wherein mining comprises mining from a first data source of the electronic medical record and mining from a second data source of the electronic medical record, the first data source comprising structured data and the second data source comprising unstructured data, the mining outputting values for the feature vector in a structured format from the first and second data sources.

5. The method of claim 3 wherein mining comprises inferring a value for each of a plurality of variables, each value inferred by probabilistic combination of probabilities associated with different possible values from different sources, the inferred values for the variables comprising the feature vector.

6. The method of claim 3 where mining comprises mining as a function of existing knowledge, guidelines, best practices, or about specific institutions regarding case management.

7. The method of claim 1 wherein generating the at least one alert comprises generating a cell phone alert, a computer alert, an alert in the workflow, or combinations thereof.

8. The method of claim 1 further comprising:
automatically scheduling a job entry in a workflow of a case manager, the job entry being for examination to avoid readmission.

9. The method of claim 1 wherein gathering the second clinical data comprises obtaining the clinical data from performance of at least one of the multiple actions, and wherein updating comprises altering a severity for the patient and reassigning the workflow based on the altered severity.

10. The method of claim 1 wherein establishing the workflow and updating the workflow comprises setting the workflow as a function of the cost factor comprising a cost of care and a reimbursement for the care of the workflow.

11. The method of claim 1 further comprising predicting, with the processor, a probability of meeting the timeline, a cost associated with meeting the timeline, and a strongest link to the probability indicating a risk of failure to meet the timeline, the strongest link being relative to links for other variables to the risk.

12. The method of claim 1 wherein establishing the workflow for the care comprises establishing the workflow for the care of the patient after a discharge and to be performed by multiple medical professionals.

13. A system for computer-based patient management for healthcare, the system comprising:
at least one memory operable to store data for a plurality of patients; and
a first processor configured to:
classify each of the patients into diagnosis-related groups based on respective data for each of the patients;
select a timeline to discharge as a function of the diagnosis-related group for each of the patients;
alter the diagnosis-related group for at least one of the patients, the altering being based on a utilization and new data not used in the classifying;
change the timeline for the one of the patients, the changing being a function of the altering; and
monitor tasks across multiple medical professionals as a function of the timeline.

14. The system of claim 13 wherein the processor is configured to mine the data including mining unstructured information, the mining providing values for the variables, the values inferred from different possible values in the data and probabilities assigned to the possible values.

15. The system of claim 13 wherein the processor is configured to generate an alert in response to the monitoring.

16. The system of claim 13 wherein the processor being configured to alter based on the utilization and new data not used in the classifying comprises the processor being configured to change the diagnosis-related group for the one patient, the changing being a function of cost of the tasks, reimbursement for the tasks, and the new data, the new data being obtained after the classifying and while the patient is being treated at a healthcare facility.

17. In a non-transitory computer readable storage medium having stored therein data representing instructions executable by a programmed processor for computer-based patient management for healthcare, the storage medium comprising instructions for:
acquiring data for a patient;
establishing, as a function of the data, first care for the patient prior to an admission to a healthcare facility;
managing, as a function of the data, second care for the patient upon the admission to the healthcare facility; and
establishing, as a function of the data, third care for the patient after discharge from the healthcare facility.

18. The non-transitory computer readable storage medium of claim 17 further comprising generating an alert, reminder, or task as part of the first care, second care, third care, or combinations thereof.

19. The non-transitory computer readable storage medium of claim 17 wherein the first care, second care, and third care are across multiple medical providers associated with different institutions, one of the different institutions comprising the healthcare facility.

20. The non-transitory computer readable storage medium of claim 17 wherein acquiring the data comprises mining unstructured information, the mining providing values for variables, the values inferred from different possible values and probabilities assigned to the possible values.

21. The non-transitory computer readable storage medium of claim 17 wherein establishing the first care comprises identifying a first cohort for the patient, and wherein managing the second care, establishing the third care, or both comprise identifying a second cohort for the patient, the second cohort different than the first cohort.

22. The non-transitory computer readable storage medium of claim 17 wherein establishing, from the data, the third care comprises predicting a risk of readmission, predicting a financial impact, and predicting a severity.

23. The non-transitory computer readable storage medium of claim 17 wherein establishing the first care, managing the second care, and establishing the third care comprise setting as a function of healthcare resource consumption.

24. The non-transitory computer readable storage medium of claim 17 further comprising:
presenting first information associated with the first care, the second care, the third care, or combinations thereof to a first user of a first role;
presenting second information associated with the first care, the second care, the third care, or combinations thereof to a second user of a second role, the second role and second information different than the first role and first information, respectively.

25. The non-transitory computer readable storage medium of claim 17 further comprising indicating a performance of a medical professional across multiple patients including the patient.
